(19) United States
(12) Patent Application Publication
(10) Pub. No.: US 2010/0305669 A1
(43) Pub. Date: Dec. 2, 2010

(54) Neck Pad

(75) Inventor: Yuichi Sasaki, Osaka (JP)

Correspondence Address: EDWARDS ANGELL PALMER & DODGE LLP, P.O. BOX 55874, BOSTON, MA, 02205, US

(73) Assignee: KOBAYASHI PHARMACEUTICAL CO., LTD, Osaka (JP)

(21) Appl. No.: 12/227,698

(22) PCT Filed: May 29, 2007
(86) PCT No.: PCT/JP2007/060903

§ 371 Date: Apr. 19, 2010

(30) Foreign Application Priority Data
May 31, 2006 (JP) 2006-152911

(51) Int. Cl. A61F 7/00

(52) U.S. Cl. 607/109; 607/112; 607/114

(57) ABSTRACT

[Problems] A neck pad capable of enhancing fitting feeling to the neck and effectively warming or cooling the neck and its vicinity.

[Means for Solving Problems] A neck pad for attaching to the neck comprising a housing unit (1) disposed at the back of the neck to house a cooling element or a heating element, and a pair of winding portions (2) connected with the opposite ends of the housing unit (1), constructed to be gradually more slender toward the tip ends, and arranged to be passed around the neck.

### 1. TECHNICAL FIELD

The present invention relates to a neck pad for use in recuperation from tiredness, or the like.

### 2. BACKGROUND ART

Bad blood circulation due to muscular contraction causes “stiffness” in the body, such as stiff neck, which is a significant health issue in daily life. With the increase of jobs in which people are required to stay seated during prolonged work periods, like computer work, many people now have chronic stiffnesses, typically in the neck/shoulder area.

Particularly, stiffness in the muscles near the head, such as the trapezius, can cause tension-type headaches. Tension-type headaches are often associated with eyestrain, lassitude, vertigo, etc., which greatly affect people's daily lives. Research revealed that tension-type headaches are often induced by stiffness in the topmost part of the trapezius located in the lower part of the occipital region (Non-Patent Document 1).

A common treatment to assuage such stiffnesses is warming muscles to promote blood circulation (Non-Patent Document 2). For example, Patent Document 1 discloses a rectangular pad for warming the neck area, using a heat-generating member or the like.
Non-Patent Document 1: Manabu Sakuta, Kincho-gata zutu no hassei-kijyo (Developmental Mechanism of Tension-type Headaches), Sinkei Sinpo (Nervous Development), 46-3 2002
Non-Patent Document 2: Ono et al. Seikeigekatekisikkan ni taisuru HP-37 no rinsho-koka (Clinical Effects of HP-37 on Orthopedic Diseases), Rinsho to kenkyu (Studies and Clinical Research) Vol. 67, No. 5
Patent Document 1: Japanese Unexamined Patent Publication No. 2003-47627

### DISCLOSURE OF THE INVENTION

### Technical Problem

However, the rectangular pad according to Patent Document 1 fits poorly on the neck. More specifically, because the neck is located adjacent to the head and shoulders, it is difficult to apply such a rectangular pad to the neck without the formation of a gap between the affected part and the pad, and thus the pad cannot perfectly fit on the neck. This occurs more often in the trapezius in the lower part of the occipital region, resulting in insufficient warming of the affected part.

A headache caused by stiffness can be assuaged by taking headache remedies, muscle relaxants etc. However, these medicines may cause sleepiness as a side effect; additionally, people are insecure about the continuous administration of such medicines.

Another type of headache, a “migraine”, caused by the vasodilation of blood vessels, is known to be relieved by decreasing the blood flow rate by cooling the neck. However, there is no disclosed art regarding efficient neck-cooling methods.

The present invention was made to solve the foregoing problems, and an object is to provide a neck pad with an improved fit on the neck, which effectively warms and cools the neck and vicinity thereof.

### Technical Solution

The present invention was made to solve the foregoing problems, and provides a neck pad to be applied to the neck, the neck pad comprising: a container portion to be disposed in the back of the neck, containing a cooling member or a heat-generating member; and a pair of wrapping portions to be wrapped around the neck, connected to the two ends of the container portion, each wrapping member being configured to gradually narrow toward its end.

The neck pad is structured so that each wrapping portion is configured to gradually narrow toward the tip. With this structure, the wrapping portion will not touch the chin etc. of the user when the container portion is disposed on the lower occipital region; therefore, the wrapping portions fit directly along the lateral sides of the neck. Accordingly, the neck pad ensures its warming/cooling effect not only for the area in contact with the pad, but also for the area from the neck to the shoulders and vicinity thereof. The neck pad thus effectively eases headaches. Other potential advantages given by the neck pad are improved blood circulation and ease of muscular contraction. The present invention is thus effective for health enhancement.

In view of the average human neck circumference, the neck pad is preferably arranged so that the direct line distance between the ends of the wrapping portions is 15 to 38 cm. With this length, the neck pad covers more than half the circumference of an average human neck, and the ends of the wrapping portions will not overlap each other. Therefore, the neck pad effectively warms/cools the neck without placing the neck under strain. Further, the user will not experience a rush of blood to the head, even after long hours of use.

The shape of the wrapping portions is not limited as long as they gradually narrow toward the respective tips. Examples of such shapes include a triangle and a sickle. Particularly, when each wrapping section is configured to be curved downward toward its tip, the wrapping portion will not touch the chin, thereby allowing the neck pad to be securely wrapped around the neck. Further, by configuring the lower end of each wrapping portion convexly to form an upward curve substantially in the shape of a circular arc at the connection of the wrapping portion and the container portion, the lower end will fall along the curve of the back to the front of the shoulder when the wrapping portion is wrapped around the neck. This further improves the fit.

The neck pad further preferably comprises a flexible substantially-U-shaped collar formed of a container portion-supporting base for supporting the container portion, and wrapping portion-supporting bases respectively connected to two sides of the container portion-supporting base to support the wrapping portions. The neck pad still further preferably comprises a U-shape collar. The collar keeps the U-shape of the neck pad, and thereby securely fixes the pad to the neck, preventing the pad from accidentally coming off the neck.

The shape of the container portion-supporting base is not limited. For example, the container portion-supporting base may be a circular arc having a central angle of 60° to 200°, and a curvature radius of 4.5 to 7.0 cm. With this shape, the neck pad will cover the area from the lower occipital region to the lateral sides of the neck, and will appropriately fit on the neck of an average person.

Around the, collar, the wrapping portion-supporting base is preferably configured substantially in a linear shape. With this structure, the wrapping portion-supporting base will not wind around the neck, and the area touching the lateral sides of the neck is reduced. Therefore, the pad will not excessively squeeze the neck, and the neck will not feel overly constricted.

To ensure an appropriate fit for an average person, an angle created by the wrapping portion-supporting base and a contact line on the connection of the container portion-supporting base and the wrapping portion-supporting base is preferably 0° to 25°.

To keep a proper fit on the neck and an appropriate tightness, the separation strength to uncouple the two ends of the wrapping portions is 0.09 to 0.39 N/cm.

To prevent the wrapping section from touching the chin and to keep the appropriate fit on the neck, an upper end of the container portion-supporting base and an upper end of the wrapping portion-supporting base are connected by creating a downward angle of 10° to 35°.

To improve the fit, the neck pad can also be structured as follows. As the second invention, the present invention provides a neck pad, containing a cooling member or a heat-generating member, to be applied to a neck, the neck pad comprising: a container portion to be disposed on the back of the neck; a pair of wrapping portions connected to the two ends of the container portion to be wrapped around the neck; a container portion-supporting base for supporting the container portion; and wrapping portion-supporting bases respectively connected to the two sides of the container portion-supporting base to support the wrapping portions, wherein the neck pad is provided with a flexible U-shape collar.

This structure keeps the U-shape of the neck pad, and thereby securely fixes the pad to the neck. With this structure, the pad ensures improved fit and therefore is prevented from accidentally coming off the neck.

The second neck pad is preferably arranged so that the direct line distance between the ends of the wrapping portions is 15 to 38 cm.

The second neck pad is preferably arranged so that each wrapping member is configured to gradually narrow toward its tip.

The second neck pad is preferably arranged so that a lower end of each wrapping portion convexly forms an upward curve substantially in a shape of a circular arc at the connection of the wrapping portion and the container portion.

The second neck pad is preferably arranged so that the container portion-supporting base is a circular arc having a central angle of 60° to 200°, and a curvature radius of 4.5 to 7.0 cm.

The second neck pad is preferably arranged so that the wrapping portion-supporting base is configured substantially in a linear shape.

The second neck pad is preferably arranged so that an angle created by the wrapping portion-supporting base and a contact line on a connection of the container portion-supporting base and the wrapping portion-supporting base is 0° to 25°.

The second neck pad is preferably arranged so that the two ends of the wrapping portions are separated from each other by a separation strength of 0.09 to 0.39 N/cm.

The second neck pad is preferably arranged so that an upper end of the container portion-supporting base and an upper end of the wrapping portion-supporting base are connected by creating a downward angle of 10° to 35°.

### EFFECT OF THE INVENTION

The neck pad of the present invention ensures an improved fit on the neck, thereby effectively warming or cooling the neck and vicinity thereof.

### BEST MODE FOR CARRYING OUT THE INVENTION

One embodiment of the neck pad according to the present invention is described below with reference to drawings. FIG. 1 is a perspective view of a neck pad according to the present invention. FIG. 2 is a development view of the neck pad.

As shown in FIG. 1, the neck pad is configured to be wrapped around the user's neck for the purpose of recuperation from tiredness etc. The neck pad is formed of a pad body 3 including a container portion 1 that is disposed behind the neck when the pad is applied to the neck, and a pair of wrapping portions 2 respectively combined with the two ends of the container portion 1 to be wrapped around a user's neck. The neck pad also has a strap-shaped connector member 4 that connects the right and left wrapping portions 2. The connector member 4 has a hook-and-loop fastener or the like, which attaches and fixes one of the left and right wrapping portions 2 to the other to connect them to each other. Further, the pad body 3 therein contains a detachable collar 5, which serves to keep the pad body 3 in a U-shape.

The following describes the pad body 3. The container portion 1 has a substantially rectangular shape, and includes a pocket 11 on a surface opposite the neck. The pocket 11 therein has a heat-generating member such as a body warmer. In view of the fit on the neck, and the warming or cooling effect etc., the long-side of the container portion 1 is preferably 5 to 16 cm, more preferably 9 to 12 cm, and the short-side is preferably 2 to 12 cm, more preferably 7 to 11 cm. In addition to the warmer mentioned above, the heat-generating member can be a hot water bottle, an anka (foot warmer), an electric foot warmer, a disposable body warmer, a hot pack heatable in microwaves or in hot water, a heat-generating gel pad, or a body warmer using a heat-generating reaction of an acetic acid sodium. In addition, the pocket 11 can also contain a cooling member such as a cooling gel pack, or a cooling pack using a reaction of urea, aluminum nitrate and water.

The pocket 11 has an opening having a fixing means (not shown) such as a hook-and-loop fastener, adhesive tape or the like, to hold the heat-generating member or a cooling member inside the pocket 11. Further, this fixing means also serves to block the opening of the pocket 11, and thereby prevents the heat-generation temperature from varying due to the influence of the peripheral air content when the pocket 11 contains a heat-generating member using oxidation such as a body warmer. This keeps the air content in the pocket 11 constant, thereby stabilizing the temperature over time.

As shown in FIG. 2, each wrapping portion 2 has a sickle-like shape with a vertical width smaller than the container portion 1. Each wrapping portion 2 is curved downward and gradually narrow toward the tip. Further, each wrapping portion 2 extends from the upper portion of each side of the container portion 1, so that a lower end of the container portion 1 protrudes downward from the pad body and a lower end 21 of the wrapping portion 2 convexly forms an upward curve substantially in the shape of a circular arc at the connection of the wrapping portion 2 and the container portion 1. In this configuration, the wrapping portion 2 is more preferably configured so that the direct line distance A to C is longer than the direct line distance B to C, as shown in FIG. 2, to avoid contact with the user's chin. The point A is the connection between an upper end of the wrapping portion 2 and the container portion 1, and the point B is the connection of the lower end 21 of the wrapping portion 2 and the container portion 1. The point C is generally the tip of the wrapping portion 2; however, as shown in FIG. 2, when the wrapping portion 2 is curved downward beyond the horizontal straight line that extends from the point B, the point C is the connection of the horizontal straight line and the tip of the wrapping portions 2. To ensure the desired fit and the heating or cooling effect, the distance T between the points B and C is preferably 5 to 11 cm, more preferably 7 to 9 cm.

An arbitrary material can be used for the surface of the pad body 3. Examples of suitable materials include synthetic fibers such as nylon, vinylon, polyester, rayon, acetate, acrylics, polyethylene, and polypropylene and polyvinyl chloride; and natural fibers such as cotton, hemp, and silk. Among them, polyester is particularly preferred because of its texture, and because it can effectively transmit the heat or coldness from the container portion 1 to the wrapping portions 2. Raising polyester fibers are more preferable.

Further, it is preferable to incorporate a highly elastic material into the pad body 3, such as urethane, sponge, or cotton, to increase the cushion effect, to support the weight of the head, and to ensure the effective transmission of the heat or coldness from the container portion 1 to the wrapping portion. The entire thickness of the elastic material is preferably 2 to 15 mm, and more preferably 5 to 12 mm. One end of the pad body has an opening (not shown) through which a collar, which is described below, is disposed inside the pad body.

The following explains the collar with reference to FIGS. 3 and 4. FIG. 3 is a plan view of the collar. FIG. 4 is a lateral view of the collar. As shown in FIG. 3, the collar 5 is formed of a flexible material into a U-shape. The collar 5 includes a container portion-supporting base 51 for supporting the container portion 1; and wrapping portion-supporting bases 52 respectively connected to the both sides of the container portion-supporting base 51 to support the wrapping portions 2. The material of the collar 5 can be selected from any flexible materials, including various resins such as an acrylonitrile-butadiene-styrene copolymer, polypropylene resin, polyethylene terephthalate, and vinyl chloride resin; various metals including stainless steel, aluminum, copper, and iron; wood; bamboo etc.

The container portion-supporting base 51 has substantially the same length as that of the container portion 1, and is configured to form a circular arc so that it fits to an appropriate extent on an area from a lower occipital region through the lateral sides of the neck. The central angle θ is 60 to 200°, and the curvature radius R is 4.5 to 7.0 cm. The central angle θ is more preferably 100° to 200°, further preferably 110 to 190°. The curvature radius R is preferably a circular arc of 4.5 to 6.5 cm, more preferably a circular arc of 4.5 to 6.0 cm.

Further, as shown in FIG. 3, each wrapping portion-supporting base 52 has a length sufficient to support the wrapping portion 2. The wrapping portion-supporting bases 52 extend substantially linearly from the both sides of the container portion-supporting base 51. Furthermore, as shown in FIG. 4, the upper end of the wrapping portion-supporting base 52 is connected to the upper end of the container portion-supporting base 51 by creating a downward angle. This oblique angle “φ” is preferably 10 to 35°, more preferably 10 to 25°, and further preferably 15 to 22°. By limiting the angle within this range, the wrapping portion will not touch the chin, and will appropriately fit on the neck. Furthermore, to ensure an appropriate fit, the angle φ created by the tangent line Q and the wrapping portion-supporting base 52 on the connection P of the two supporting bases is preferably 0 to 25°, more preferably 5 to 20°, further preferably 10 to 20°.

Moreover, to improve the fit by making the collar press the lateral sides of the neck to an appropriate extent, the length S of the wrapping portion-supporting base 52, which is defined as a length from its tip to the connection point with the container portion-supporting base 51, is preferably 3 to 10 cm, and more preferably 5 to 8 cm. The height H of the collar 5 is not limited as long as the collar 5 can support the pad body 3; however, the height H is preferably 1 to 10 cm, more preferably 2 to 8 cm, and further preferably 3 to 5 cm to ensure that the collar 5 presses the lateral sides of the neck to an appropriate extent and thereby attains the improved fit.

In addition to the appropriate fit, the collar 5 must have a certain flexibility to ensure the transmission of the heat and coldness from the lower occipital region to the lateral sides of the neck. Here, the recovery force that is exerted when the respective tips of the wrapping portion-supporting bases 52 are separated away from each other in the direction shown by the arrow F in FIG. 4 is referred to as a separation strength; and the material, size etc. of the collar is appropriately selected so that the separation strength falls within the predetermined range. For example, the separation strength is measured as follows using an Autograph AGS-500D (Shimadzu Corporation). The tip of one of the wrapping portion-supporting bases is fixedly attached to the Autograph AGS-500D, and the tip of the other wrapping portion-supporting base is bent away from the aforementioned tip at 300 mm/min. Then, the recovery force is measured. In the present embodiment, the separation strength is preferably 0.09 to 0.39 N/cm, more preferably 0.13 to 0.36 N/cm. If the separation strength is less than 0.09 N/cm. the sufficient fit is not obtained, and the pad may come off the neck. If the separation strength is more than 0.39 N/cm, the pressure on the neck becomes excessively high, and the user will feel excessive tightness around the neck. Particularly, when the wrapping portion-supporting base 52 extends linearly as in the case above, the separation strength is preferably 0.20 to 0.35 N/cm. The foregoing range of the separation strength is also suitable for the neck pad, regardless of whether the neck pad has the collar or not.

The neck pad having the aforementioned structure is used in the following manner. In accordance with intended use, the heat-generating member is placed in the pocket 11 of the container portion 1. Then, the tips of the wrapping portions 2 are pushed outward to set the neck pad on the neck, as shown in FIG. 5. As a result, the container portion 1 comes in contact with the back of the neck, and heat is effectively transmitted to the area from the lower occipital region to the lateral sides of the neck, thereby relieving the stiffness in the trapezius. This can ease tension-type headaches. In the case of a migraine caused by the vasodilation of blood vessels, a cooling member is placed in the container portion 1. The pad containing the cooling member will cool the neck and decrease the blood flow rate, thereby relieving the pain. As described above, since the collar 5 is formed into a U-shape and given a predetermined separation strength, the pad is fixed to the neck. To further secure the fixation of the pad, a connector member 4 that combines the two wrapping portions 2 together may be provided.

As described, according to the neck pad of the present embodiment, each wrapping portion is configured to gradually narrow toward the tip. With this configuration, the wrapping portion 2 will not touch the chin of the user when the container portion 1 is disposed on the lower occipital region; therefore, the wrapping portions 2 fit on the neck along the lateral sides of the neck. The neck pad of the present embodiment thus fits better on the neck, and ensures superior comfort. Moreover, the neck pad ensures a warming/cooling effect not only for the area in contact with the pad, such as the trapezius and the lateral sides of the neck, but also for the trapezius from the neck to the shoulders, and the vicinity thereof. Therefore, it effectively eases headaches; moreover, improved blood circulation and ease of muscular contraction can also be expected. The present invention is thus effective for health enhancement.

In addition, the wrapping portion-supporting base 52 in the collar 5 linearly extends from the container portion-supporting base 51. With this configuration, only a part of the collar 5 comes in contact with the neck, preventing the collar 5 from excessively pressing the neck. With this effect, an appropriate fit and comfort can be obtained at the same time.

The present invention should not be narrowly interpreted within the limits of the embodiment discussed in the foregoing detailed explanation, but rather may be applied in many variations, provided such variations do not exceed the scope of the present invention. For example, the shape of each wrapping portion 2 is not limited to the foregoing example above, as long as it is configured to gradually narrow to the tip so that the wrapping portion 2 will not touch the chin of the user. It is possible to form a wrapping portion 2 into a triangle, as shown in FIG. 6(a) and FIG. 6(b). Further, as shown in FIG. 6(c), the wrapping portion 2 may be configured to have the same vertical height as that of the container portion 1.

Likewise, the collar 5 may have a shape other than the one described above. For example, as shown in FIG. 7(a), the collar 5 may be configured such that the wrapping portion-supporting bases 52 are in parallel with the tangent line Q on the connection P of the two supporting bases 51 and 52. Alternatively, as shown in FIG. 7(b), the wrapping portions 2 may be curved. In this case, however, the contact area of the neck and the wrapping portion-supporting bases 52 increases, and some arrangement is required to reduce the pressure on the neck. To solve this problem, it is preferable to set the separation strength to 0.13 to 0.20N/cm. However, if the pad itself has this range of separation strength, or if the pad is fixed by the connector member 4, the collar can be omitted.

The following describes the Example of the present invention. However, the present invention is not limited to these this Examples.

### Example

Five types of neck pads, detailed in Tables 1 to 5, were tested to examine the fit, wearing comfort, warming effect, and headache relieving effect.

In this example, the neck pad has a sickle-like shape, and is configured such that the lower end of the container portion (10×10 cm) protrudes downward from the pad body, and each wrapping portion is curved downward and gradually narrow toward the end, as shown in FIG. 2 (in FIG. 2, the distance between A and B is 7 cm).

The neck pad in Comparative Example 1 has a container portion of 10×10 cm, and a rectangle wrapping portion (7×10.5 cm).

The collar was formed of an acrylonitrile-butadiene-styrene copolymer. The height of the collar was 3.5 cm both in the Example and the Comparative Example. In Tables 1 to 5, a “U-shape” collar denotes a collar having a linear wrapping portion-supporting base. A “Curved-shape” collar denotes a collar having a wrapping portion-supporting base curved with the same curvature radius as that of the container portion-supporting base and in which the direct line distance between the right and left wrapping portions is 7 cm. “Angle 1” represents an angle (“Angle φ” in FIG. 3) created by the container portion-supporting base and a tangent line on the connection of the container portion-supporting base and the wrapping portion-supporting base, “Angle” 2 represents a downward angle (“Angle φ” in FIG. 4) between the upper end of the wrapping portion-supporting base 52 and the upper end of the container portion-supporting base 51.

The separation strength was measured as follows using Autograph AGS-500D (Shimadzu Corporation). The tip of one of the wrapping portion-supporting bases was fixedly attached to the Autograph AGS-500D, and the tip of the other wrapping portion-supporting base was bent away from the aforementioned tip at 300 mm/min. Then, the recovery force was measured.

The fit on the neck, wearing comfort, warming effect, and headache-relieving effect were tested as follows.

### Fit Test

20 test subjects suffering from tension-type headaches used the neck pads for six consecutive hours, and evaluated the fit (the fit on the lower occipital region and the lateral sides of the neck) with five-grade scales. The evaluations were multiplied by the number of people to find a comprehensive score. The following are the five-grade scales.

5 Fit was good

### 4 Fit was OK

3 No opinion
2 Fit was insufficient
1 Fit was poor

### Wearing Comfort Test

20 test subjects suffering from tension-type headaches used the neck pads for six consecutive hours, and evaluated the wearing comfort (tightness) with a five-grade scale. The evaluations were multiplied by the number of people to find a comprehensive score. The following is the five-grade scale.

### 5 Comfortable

4 Fairly comfortable
3 No opinion
2 A little uncomfortable

### 1 Uncomfortable

### Warming Effect

20 test subjects suffering from tension-type headaches used the neck pads for six consecutive hours, and evaluated the warming effect (warmth in the trapezius and the lateral sides of the neck) with a five-grade scale. The evaluations were multiplied by the number of people to find a comprehensive score. The following is the five-grade scale.

### 5 Pleasant

4 Fairly pleasant
3 No opinion
2 A little unpleasant

### 1 Unpleasant

### Headache-Relieving Effect

20 test subjects suffering from tension-type headaches used the neck pads for six consecutive hours, and evaluated the headache-relieving effect with a five-grade scale. The evaluations were multiplied by the number of people to find a comprehensive score. The following is the five-grade scale.

### 5 Relieved

4 A little better
3 No opinion
2 No significant change
1 No change

TABLE 1

| | | | | | | | | | | | Compara- |
|---|---|---|---|---|---|---|---|---|---|---|---|
| | | | | | | | | | | | tive |
| | | | Examples | | | | | | | | Examples |
| | | | 1 | 2 | 3 | 4 | 5 | 6 | 7 | 8 | 1 |
| Structure | Direct Line Distance between | | 12 | 15 | 22 | 31 | 31 | 31 | 38 | 40 | 31 |
| of | the Ends of the Wrapping | | | | | | | | | | |
| Pad | Sections | | | | | | | | | | |
| | Collar | | Provided | Provided | Provided | Provided | Provided | Not | Provided | Provided | Provided |
| | | | | | | | | Provided | | | |
| | Shape | Shape | U-Shape | U-Shape | U-Shape | U-Shape | Curved- | — | U-Shape | U-Shape | U-Shape |
| | of | | | | | | Shape | | | | |
| | Collar | Central Angle of | 180 | 180 | 180 | 180 | 180 | — | 180 | 180 | 180 |
| | | Holder Portion | | | | | | | | | |
| | | Supporting Base | | | | | | | | | |
| | | Curvature Radius of | 5 | 5 | 5 | 5 | 5 | — | 5 | 5 | 5 |
| | | Holder Portion | | | | | | | | | |
| | | Angle 1 | 15 | 15 | 15 | 15 | — | — | 15 | 15 | 15 |
| | | Separation Strength | 0.22 N/cm | 0.22 N/cm | 0.22 N/cm | 0.22 N/cm | 0.22 N/cm | — | 0.22 N/cm | 0.22 N/cm | 0.22 N/cm |
| | | Angle 2 | 17 | 17 | 17 | 17 | 17 | — | 17 | 17 | 17 |

| Test | Fit on the Neck | 74 | 82 | 87 | 97 | 95 | 84 | 84 | 81 | 51 |
|---|---|---|---|---|---|---|---|---|---|---|
| Result | Wearing Comfort | 73 | 83 | 88 | 98 | 84 | 88 | 84 | 83 | 53 |
| | Warming Effect | 74 | 80 | 85 | 97 | 90 | 86 | 80 | 87 | 55 |
| | Headache Relieving | 71 | 75 | 90 | 94 | 92 | 86 | 85 | 84 | 50 |
| | Effect | | | | | | | | | |

TABLE 2

| | | | Examples | | | | | | | |
|---|---|---|---|---|---|---|---|---|---|---|
| | | | 9 | 10 | 11 | 12 | 4 | 13 | 14 | 15 |
| Structure | Direct Line Distance between | | 31 | 31 | 31 | 31 | 31 | 31 | 31 | 31 |
| of | the Ends of the Wrapping | | | | | | | | | |
| Pad | Sections | | | | | | | | | |
| | Collar | | Provided | Provided | Provided | Provided | Provided | Provided | Provided | Provided |
| | Shape | Shape | Curved | U-Shape | U-Shape | U-Shape | U-Shape | U-Shape | U-Shape | U-Shape |
| | of | | Shape | | | | | | | |
| | Collar | Central Angle of | 60 | 55 | 80 | 120 | 180 | 200 | 220 | 180 |
| | | Holder Portion | | | | | | | | |
| | | Supporting Base | | | | | | | | |
| | | Curvature Radius of | 4 | 5 | 5 | 5 | 5 | 5 | 5 | 8 |
| | | Holder Portion | | | | | | | | |
| | | Angle 1 | — | 0 | 15 | 15 | 15 | 15 | 5 | 15 |
| | | Separation Strength | 0.22 N/cm | 0.22 N/cm | 0.22 N/cm | 0.22 N/cm | 0.22 N/cm | 0.22 N/cm | 0.22 N/cm | 0.22 N/cm |
| | | Angle 2 | 17 | 17 | 17 | 17 | 17 | 17 | 17 | 17 |

| Test | Fit on the Neck | 81 | 83 | 85 | 95 | 97 | 92 | 83 | 80 |
|---|---|---|---|---|---|---|---|---|---|
| Result | Wearing Comfort | 85 | 89 | 90 | 95 | 98 | 91 | 90 | 88 |
| | Warming Effect | 80 | 82 | 86 | 94 | 97 | 90 | 81 | 81 |
| | Headache Relieving | 82 | 84 | 87 | 92 | 94 | 87 | 85 | 83 |
| | Effect | | | | | | | | |

TABLE 3

| | | | Examples | | | | | |
|---|---|---|---|---|---|---|---|---|
| | | | 16 | 17 | 18 | 4 | 19 | 20 |
| Structure | Direct Line Distance between | | 31 | 31 | 31 | 31 | 31 | 31 |
| of | the Ends of the Wrapping | | | | | | | |
| Pad | Sections | | | | | | | |
| | Collar | | Provided | Provided | Provided | Provided | Provided | Provided |
| | Shape | Shape | U-Shape | U-Shape | U-Shape | U-Shape | U-Shape | U-Shape |
| | of | Central Angle of | 180 | 180 | 180 | 180 | 180 | 180 |
| | Collar | Holder Portion | | | | | | |
| | | Supporting Base | | | | | | |
| | | Curvature Radius of | 5 | 5 | 5 | 5 | 5 | 5 |
| | | Holder Portion | | | | | | |
| | | Angle 1 | 30 | 25 | 20 | 15 | 0 | −5 |
| | | Separation Strength | 0.22 N/cm | 0.22 N/cm | 0.22 N/cm | 0.22 N/cm | 0.22 N/cm | 0.22 N/cm |
| | | Angle 2 | 17 | 17 | 17 | 17 | 17 | 17 |

| Test | Fit on the Neck | 87 | 92 | 95 | 97 | 82 | 80 |
|---|---|---|---|---|---|---|---|
| Result | Wearing Comfort | 75 | 87 | 91 | 98 | 83 | 77 |
| | Warming Effect | 84 | 92 | 95 | 97 | 93 | 78 |
| | Headache Relieving | 88 | 92 | 95 | 94 | 90 | 89 |
| | Effect | | | | | | |

TABLE 4

| | | | Examples | | | | | | | |
|---|---|---|---|---|---|---|---|---|---|---|
| | | | 21 | 22 | 23 | 24 | 4 | 25 | 26 | 27 |
| Structure | Direct Line Distance between | | 31 | 31 | 31 | 31 | 31 | 31 | 31 | 31 |
| of | the Ends of the Wrapping | | | | | | | | | |
| Pad | Sections | | | | | | | | | |
| | Collar | | Provided | Provided | Provided | Provided | Provided | Provided | Provided | Provided |
| | Shape | Shape | U-Shape | U-Shape | U-Shape | Curved | U-Shape | U-Shape | U-Shape | U-Shape |
| | of | | | | | Shape | | | | |
| | Collar | Central Angle of | 180 | 180 | 180 | 180 | 180 | 180 | 180 | 180 |
| | | Holder Portion | | | | | | | | |
| | | Supporting Base | | | | | | | | |
| | | Curvature Radius of | 5 | 5 | 5 | 5 | 5 | 5 | 5 | 5 |
| | | Holder Portion | | | | | | | | |
| | | Angle 1 | 15 | 15 | 15 | — | 15 | 15 | 15 | 15 |
| | | Separation Strength | 0.05 N/cm | 0.09 N/cm | 0.13 N/cm | 0.13 N/cm | 0.22 N/cm | 0.36 N/cm | 0.39 N/cm | 0.45 N/cm |
| | | Angle 2 | 17 | 17 | 17 | 17 | 17 | 17 | 17 | 17 |

| Test | Fit on the Neck | 88 | 92 | 90 | 96 | 97 | 96 | 95 | 90 |
|---|---|---|---|---|---|---|---|---|---|
| Result | Wearing Comfort | 80 | 88 | 90 | 95 | 98 | 84 | 80 | 72 |
| | Warming Effect | 75 | 84 | 94 | 95 | 97 | 93 | 86 | 80 |
| | Headache Relieving | 75 | 80 | 92 | 94 | 94 | 92 | 86 | 80 |
| | Effect | | | | | | | | |

TABLE 5

| | | | Examples | | | | | |
|---|---|---|---|---|---|---|---|---|
| | | | 28 | 29 | 4 | 30 | 31 | 32 |
| Structure | Direct Line Distance between | | 31 | 31 | 31 | 31 | 31 | 31 |
| of | the Ends of the Wrapping | | | | | | | |
| Pad | Sections | | | | | | | |
| | Collar | | Provided | Provided | Provided | Provided | Provided | Provided |
| | Shape | Shape | U-Shape | U-Shape | U-Shape | U-Shape | U-Shape | U-Shape |
| | of | Central Angle of | 180 | 180 | 180 | 180 | 180 | 180 |
| | Collar | Holder Portion | | | | | | |
| | | Supporting Base | | | | | | |
| | | Curvature Radius of | 5 | 5 | 5 | 5 | 5 | 5 |
| | | Holder Portion | | | | | | |
| | | Angle 1 | 15 | 15 | 15 | 15 | 15 | 15 |
| | | Separation Strength | 0.22 N/cm | 0.22 N/cm | 0.22 N/cm | 0.22 N/cm | 0.22 N/cm | 0.22 N/cm |
| | | Angle 2 | 5 | 10 | 17 | 25 | 35 | 40 |

| Test | Fit on the Neck | 75 | 88 | 97 | 94 | 90 | 83 |
|---|---|---|---|---|---|---|---|
| Result | Wearing Comfort | 72 | 87 | 98 | 95 | 91 | 86 |
| | Warming Effect | 80 | 85 | 97 | 92 | 90 | 88 |
| | Headache Relieving | 80 | 83 | 94 | 92 | 90 | 85 |
| | Effect | | | | | | |

The body surface temperature before and after using the neck pad according to Example 4 and the neck pad according to the Comparative Example 1 was measured using thermography (device: Thermo Tracer TH9100MV (NEC San-ei). A test subject naked from the waist up was exposed to an environment of 25° C. and 60% humidity, for 15 minutes, and had a thermography image taken. Then, the test subject wore the pad according to Example 1 and the pad according to Comparative Example 1. 30 minutes after wearing a test pad, the test subject naked from the waist up had a thermography image taken. FIG. 8 (Example 4) and FIG. 9 (Comparative Example 1) show the results.

After comparing the neck pad of Example, which includes the wrapping portions connected to both sides of the container portion with each end gradually narrowing toward the tip, and the pad of Comparative Example 1, which does not contain the wrapping portions, it is clear that the neck pad of Example was superior in its fit on the neck, wearing comfort, warming effect, and headache-relieving effects over the neck pad of Comparative Example 1.

According to the results in Table 1, the direct line distance between the right and left wrapping portions is preferably 15 to 38 cm. According to the results in Table 2, the container portion-supporting base of the collar is preferably a circular arc having a central angle of 60 to 200°, and a curvature radius R of 4.5 to 7.0 cm. According to the results in Table 3, “Angle 1” (“Angle φ” in FIG. 3) is preferably 0 to 25°. According to the results in Table 4, the separation strength to separate the ends of the wrapping sections is preferably 0.09 to 0.39 N/cm. According to the results in Table 5, “Angle 2” (“Angle φ” in FIG. 4) is preferably 10 to 325°.

Further, for example, the comparison between Example 4 or 5, and Example 6 shows that the neck pad preferably includes a collar. The comparison between Examples 4 and 5 shows that the collar preferably has a U-shape. In other words, the collar preferably includes substantially linear wrapping portion-supporting bases, given that the central angle and curvature radius of the container portion-supporting base, the separation strength, and “Angle 2” are unchanged.

Further, FIGS. 8 and 9 show that the neck pad of the Example has a superior heating effect over the neck pad of Comparative Example 1. Particularly, using the neck pad of the Example, the pad not only warms the trapezius and lateral sides of the neck coming into contact with the pad, it can also warm the trapezius from the neck to the shoulders, and the vicinity thereof, thereby effectively facilitating blood circulation, which relieves muscle contraction and headaches.

### BRIEF DESCRIPTION OF THE DRAWINGS

FIG. 1 is a perspective view of a neck pad according to the First Embodiment of the present invention.

FIG. 2 is a development view of the neck pad of FIG. 1.

FIG. 3 is a plan view of a collar according to the First Embodiment, provided in the neck pad of FIG. 1.

FIG. 4 is a lateral view of the collar of FIG. 3.

FIG. 5 is a perspective view showing how to wear the neck pad according to the present embodiment.

FIG. 6 is a development view of a neck pad according to another embodiment of the present invention.

FIG. 7 is a plan view of a collar according to another embodiment, provided in the neck pad of the present invention.

FIG. 8 is a thermography image of a user wearing the neck pad according to Example 4.

FIG. 9 is a thermography image of a user wearing the neck pad according to Comparative Example 1.

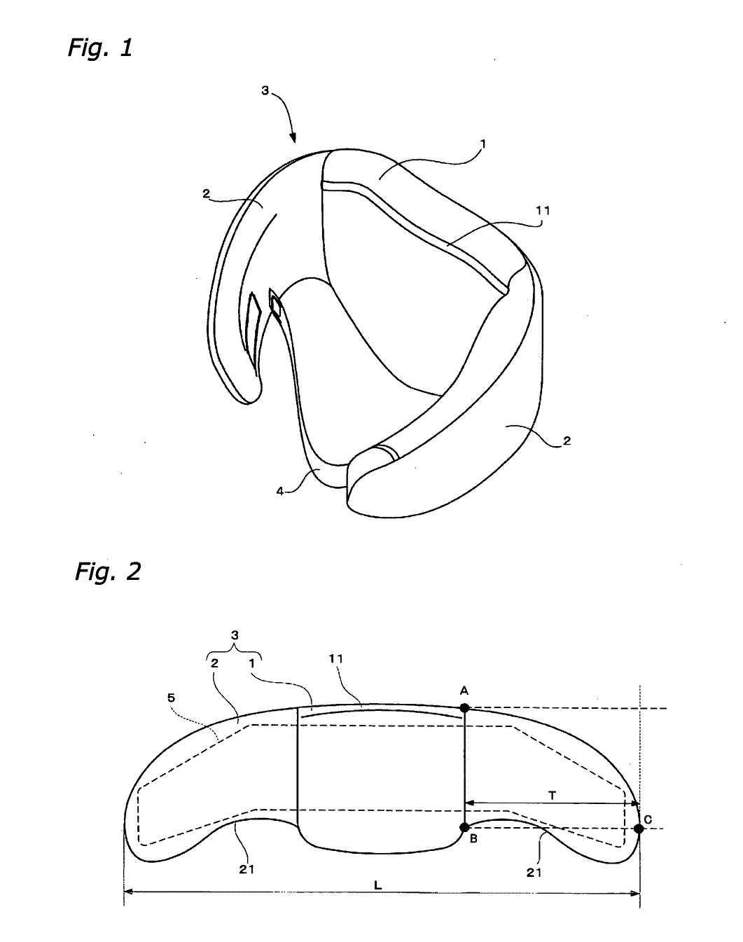

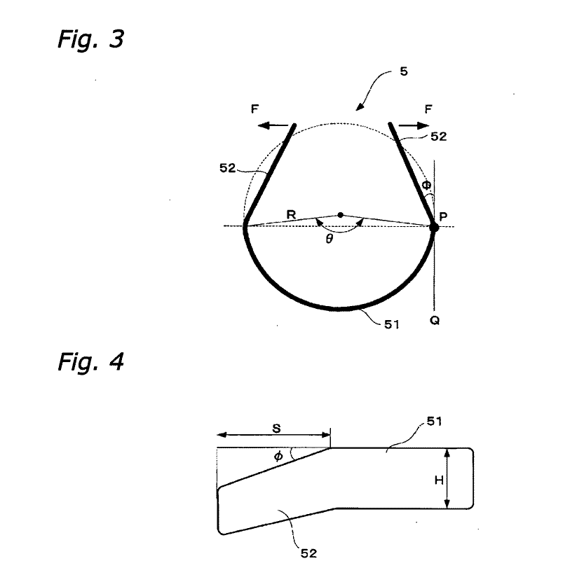

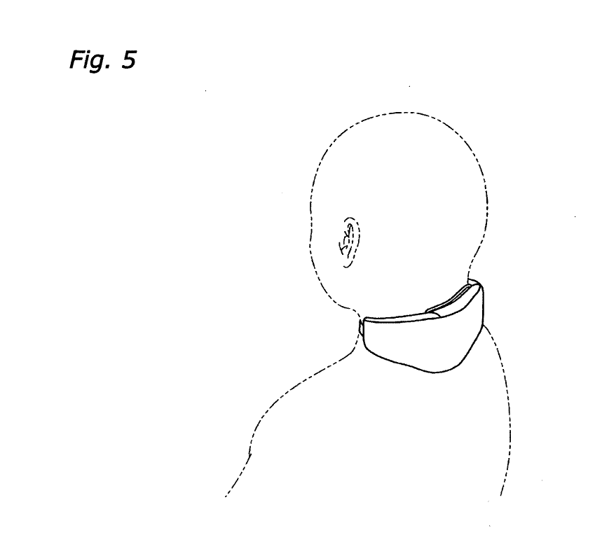

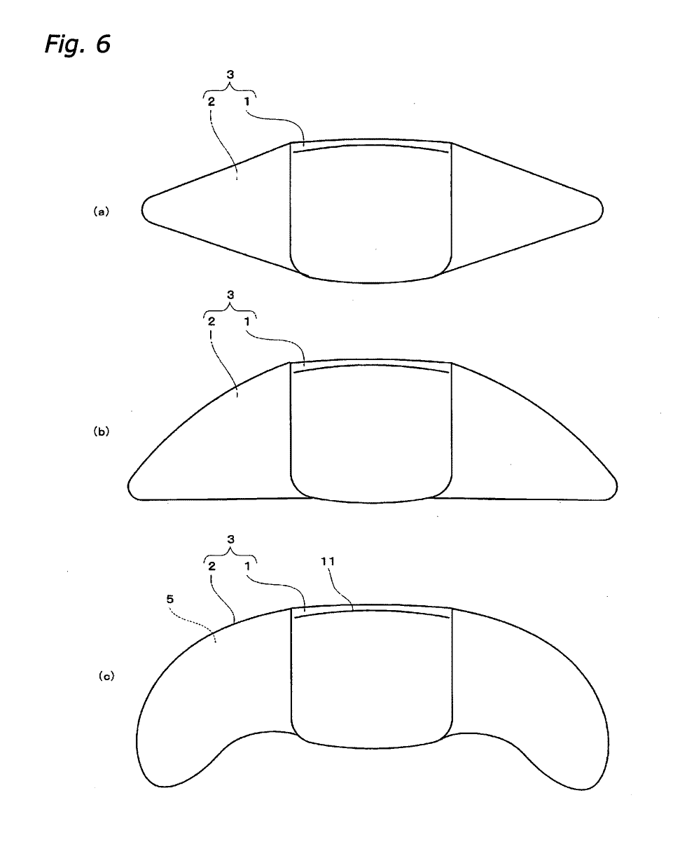

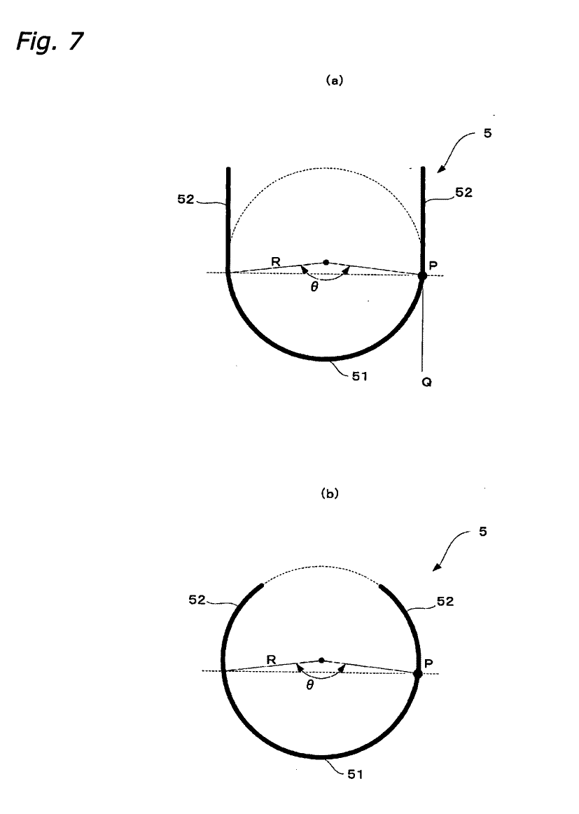

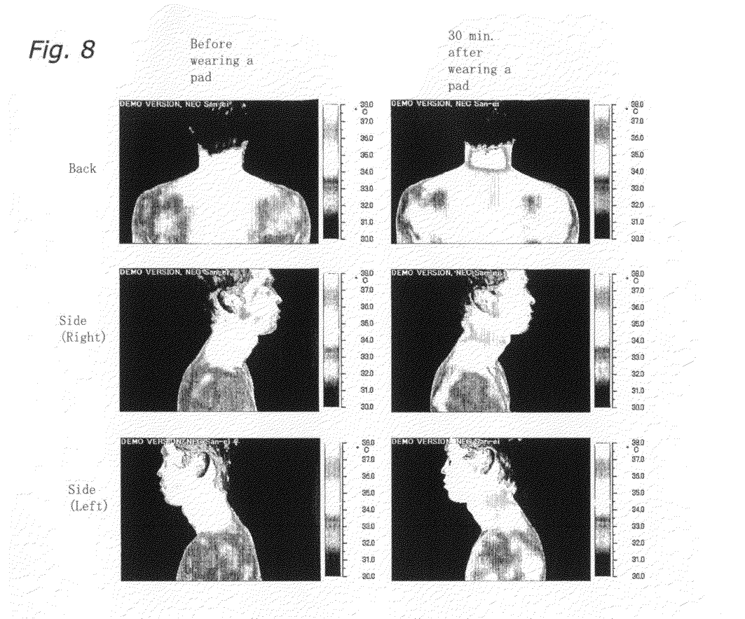

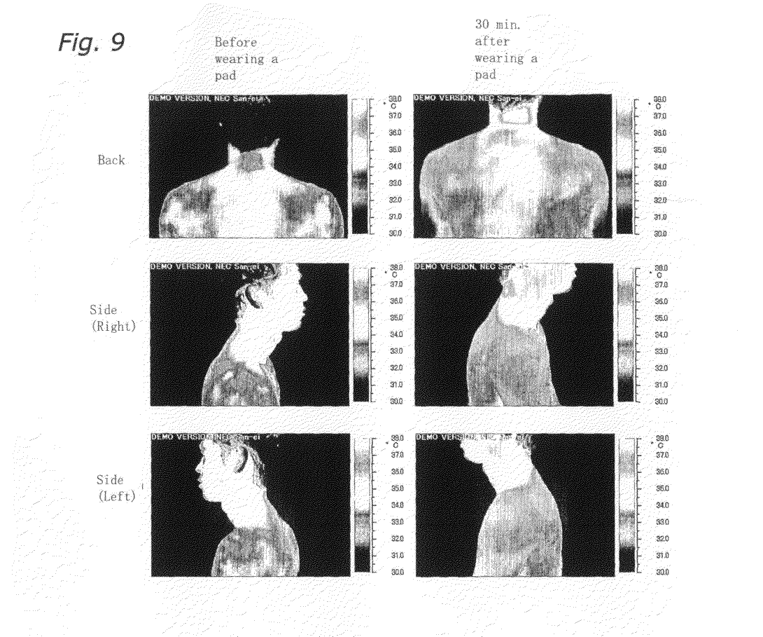

### REFERENCE NUMERALS

1 Container portion
2 Wrapping portion

1. A neck pad to be applied to a neck, comprising:
a container portion to be disposed on the back of the neck, containing a cooling member or a heat-generating member; and
a pair of wrapping portions to be wrapped around the neck, connected to the two ends of the container portion, each wrapping member being configured to gradually narrow toward its end.

2. The neck pad according to claim 1, wherein the direct line distance between the ends of the wrapping portions is 15 to 38 cm.

3. The neck pad according to claim 1, wherein a lower end of each wrapping portion convexly forms an upward curve substantially in the shape of a circular arc at a connection of the wrapping portion and the container portion.

4. The neck pad according to claim 1, further comprising a flexible substantially-U-shaped collar formed of, a container portion-supporting base for supporting the container portion, and a wrapping portion-supporting bases respectively connected to the two ends of the container portion-supporting base to support the wrapping portions.

5. The neck pad according to claim 4, wherein the container portion-supporting base is a circular arc having a central angle of 60° to 200°, and a curvature radius of 4.5 to 7.0 cm.

6. The neck pad according to claim 4, wherein each wrapping portion-supporting base is substantially linear.

7. The neck pad according to claim 4, wherein an angle created by the wrapping portion-supporting base and a contact line on a connection of the container portion-supporting base and the wrapping portion-supporting base is 0° to 25°.

8. The neck pad according to claim 1, wherein the two ends of the wrapping portions are separated from each other by a separation strength of 0.09 to 0.39 N/cm.

9. The neck pad according to claim 4, wherein an upper end of the container portion-supporting base and an upper end of the wrapping portion-supporting base are connected by creating a downward angle of 10° to 35°.

10. The neck pad according to claim 2, wherein a lower end of each wrapping portion convexly forms an upward curve substantially in the shape of a circular arc at a connection of the wrapping portion and the container portion.

11. The neck pad according to claim 2, further comprising a flexible substantially-U-shaped collar formed of a container portion-supporting base for supporting the container portion, and a wrapping portion-supporting bases respectively connected to the two ends of the container portion-supporting base to support the wrapping portions.

12. The neck pad according to claim 3, further comprising a flexible substantially-U-shaped collar formed of a container portion-supporting base for supporting the container portion, and a wrapping portion-supporting bases respectively connected to the two ends of the container portion-supporting base to support the wrapping portions.

13. The neck pad according to claim 5, wherein each wrapping portion-supporting base is substantially linear.

14. The neck pad according to claim 5, wherein an angle created by the wrapping portion-supporting base and a contact line on a connection of the container portion-supporting base and the wrapping portion-supporting base is 0° to 25°.

15. The neck pad according to claim 6, wherein an angle created by the wrapping portion-supporting base and a contact line on a connection of the container portion-supporting base and the wrapping portion-supporting base is 0° to 25°.

16. The neck pad according to claim 3, wherein the two ends of the wrapping portions are separated from each other by a separation strength of 0.09 to 0.39 N/cm.

17. The neck pad according to claim 5, wherein an upper end of the container portion-supporting base and an upper end of the wrapping portion-supporting base are connected by creating a downward angle of 10° to 35°.

18. The neck pad according to claim 6, wherein an upper end of the container portion-supporting base and an upper end of the wrapping portion-supporting base are connected by creating a downward angle of 10° to 35°.

19. The neck pad according to claim 7, wherein an upper end of the container portion-supporting base and an upper end of the wrapping portion-supporting base are connected by creating a downward angle of 10° to 35°.

20. The neck pad according' to claim 8, wherein an upper end of the container portion-supporting base and an upper end of the wrapping portion-supporting base are connected by creating a downward angle of 10° to 35°.
